(19) United States
(12) Patent Application Publication
(10) Pub. No.: US 2023/0129886 A1
(43) Pub. Date: Apr. 27, 2023

(54) SYSTEMS AND METHODS OF BLOOD-TYPE PREDICTIVE ANALYSIS

(71) Applicant: Joauna Z. Carter, Richmond, TX (US)

(72) Inventor: Joauna Z. Carter, Richmond, TX (US)

(21) Appl. No.: 17/509,944

(22) Filed: Oct. 25, 2021

(51) Int. Cl. G16B 20/20; A61B 5/00

(52) U.S. Cl. CPC G16B 20/20; A61B 5/7275; A61B 2503/02

(57) ABSTRACT

Computer systems and methods of predictive analysis can be instrumental when expecting parents want to know their child's blood type. In some embodiments, the system described herein may include a processor coupled to memory, wherein the processor is operable to receive information about each parent's blood type, including the dominant-recessive allele pairs. This system can generate a first matrix of a plurality of dominant-recessive allele pairs corresponding to both parents including statistics for all combinations of the dominant-recessive allele pairs. Further, the system can generate a second matrix for all possible parental blood type pairings including the relative weights of probability associated with each blood type. In some embodiments, the system can generate a row vector specific to both parent's blood type. As output, the system can generate and display a probability rate corresponding to each possible blood type of a child and blood donor matching.

### BACKGROUND

Blood, as a fluid, delivers nutrients and oxygen to cells, while transporting waste products, such as cholesterol and proteins, away from the same. It can be classified into categories of blood types. These are inherited from a person's parents. Each person has two sets of chromosomes, one from their mother and one from their father. These chromosomes determine our blood type. Particularly, blood types are determined by the presence of antigens on a person's cells. Antigens are components of bacteria or viruses that can cause an illness if they find their way into the body. Whether A, B, O or Rh, these blood types determine the medication that a person's body can absorb, the strength of their immune system, how quickly they will age and even hair color. Blood type can also influence how certain chemicals move through the body's systems, which, as a result, can impact how healthy a person will be later in life.

Knowing a child's blood type can also help the parents to protect them from getting certain diseases and infections early in life, which could possibly make them severely ill. In other words, a child's blood makeup impacts what diseases they may be prone to and how easily they can spread the same.

In general, blood type is usually determined by a series of laboratory tests during infancy. There are many different factors that are used in the process of determining blood type. Particularly, a medical professional can assess the child's fingerprints and place a few drops of blood on a test strip. Some physicians also consider the varying differences of certain factors, such as hair color, eye color, genetic material (amniotic fluid), and infections. Although rare in practice, genetic information can be obtained from grandparents. Most labs using this technique rely on interviews between the patient, parents and the physician, in addition to reviewing medical records to generate an accurate profile of the patient.

Although these methods exist they are not easily calculated. Further, the parents must wait until after a child is born to perform most of these tests. Moreover, most people do not have an extensive knowledge base of biology to understand the significance of detecting the blood type or how blood type can be estimated.

It is within this context that the embodiments arise.

### SUMMARY

Embodiments of a system and method for blood type predictive analysis is provided. It should be appreciated that the present embodiment can be implemented in numerous ways, such as a process, an apparatus, a system, a device, or a method. Several inventive embodiments are described below.

In some embodiments, a system and method for blood type predictive analysis is provided. In particular, systems and methods of predictive analysis can be instrumental when expecting parents want to know their child's blood type. In some embodiments, the system described herein may include a blood type predictive analysis agent that couples to receive information about each parent's blood type including the dominant-recessive allele pairs. This predictive analysis agent can generate a first matrix of a plurality of dominant-recessive allele pairs corresponding to both parents including statistics for all combinations of the dominant-recessive allele pairs. For example, the blood type predictive analysis agent can detect the specific number of dominant-recessive allele pairs associated with each parent and assign each to a respective row or column, wherein one parent corresponds to the rows and one parent corresponds to the columns. Next in deriving the first matrix, the predictive analysis agent can apply each element of the first matrix to a Punnett square table and detect a count associated with each blood type, whereby each element represents the combined allele pairing for the column and row. Further, the blood type predictive analysis agent can generate a row vector specific to both parent's blood type that includes the relative weights of probability associated with each blood type, based upon the first matrix. As output, the system can generate and display a probability rate corresponding to each possible blood type of a child and the associated blood donor matching. In some embodiments, the blood type predictive analysis agent may generate the first matrix such that it includes all possible combinations for any parent blood type coupling. In this embodiment, the predictive analysis agent can generate a second matrix of all possible parental blood type pairings including the relative weights of probability associated with each blood type. It is from indexing this second matrix that the predictive analysis agent can identify a row vector having relative weights associated with each blood type for each parent using the first blood type and second blood type associated with the parents. The blood type predictive analysis agent can use these relative weights to generate a probability rate for each corresponding blood type and blood donor.

In some embodiments, a blood type predictive analysis system may include a processor coupled to a memory; wherein the processor is operable to receive user input of a first blood type associated with a first parent and a second blood type associated with a second parent, wherein the first blood type and second blood type each includes a dominant-recessive allele pair. Next, the processor can be operable to generate a first matrix of a plurality of dominant-recessive allele pairs corresponding to the first parent and the second parent. The processor may be further operable to generate a row vector, having relative weights associated with each blood type, based upon the first matrix. Additionally, the processor may be operable to sum the row vector to generate a total value and to divide each element of the row vector by the total value to generate a resultant for each blood type. From this resultant, the processor may be operable to generate a probability rate for each corresponding blood type, by multiplying each resultant by 100. Furthermore, the processor of the blood type predictive analysis system can display the probability rate corresponding to each possible blood type of a child and the associated blood donor matching pairings.

In some embodiments, a tangible, non-transitory, computer-readable media having instructions whereupon which, when executed by a processor, causes the processor to perform the blood type predictive analysis method described herein. The method of blood type predictive analysis may include receiving user input of a first blood type associated with a first parent and receiving user input of a second blood type associated with a second parent, wherein the first blood type and second blood type each includes a dominant-recessive allele pair. The method may further include generating a first matrix of a plurality of dominant-recessive allele pairs corresponding to the first parent and the second parent. For example, the system can detect the specific number of dominant-recessive allele pairs associated with each parent and assign each to a respective row or column, wherein one parent corresponds to the rows and one parent corresponds to the columns. Next in deriving the first matrix, the system can apply each element of the first matrix to a Punnett square table and detect a count associated with each blood type, whereby each element represents the combined allele pairing for the column and row. The method may further include generating a row vector, having relative weights associated with each blood type, based upon the first matrix. Additionally, the method may include summing the row vector to generate a total value, wherein each element of the row vector can be divided by the total value to generate a resultant for each blood type. Moreover, the method may include multiplying each resultant by 100 to generate a probability rate for each corresponding blood type. As output, the method may include displaying the probability rate corresponding to each possible blood type of a child and each associated blood donor matching. In some embodiments, the method may generate the first matrix such that it includes all possible combinations for any parent blood type coupling. In this embodiment, the method can generate a second matrix of all possible parental blood type pairings including the relative weights of probability associated with each blood type. The method may further include indexing the second matrix using the first blood type and second blood type to identify a row vector having relative weights associated with each blood type for each parent. These relative weights may be used to generate a probability rate for each corresponding blood type and blood donor.

Other aspects and advantages of the embodiments will become apparent from the following detailed description taken in conjunction with the accompanying drawings which illustrate, by way of example, the principles of the described embodiments.

### BRIEF DESCRIPTION OF THE DRAWINGS

The described embodiments and the advantages thereof may best be understood by reference to the following description taken in conjunction with the accompanying drawings. These drawings in no way limit any changes in form and detail that may be made to the described embodiments by one so skilled in the art without departing from the spirit and scope of the described embodiments.

FIG. 1 is a system diagram of an exemplary network incorporating the systems and methods of blood type predictive analysis, in accordance with some embodiments.

FIG. 2 is a block diagram of an exemplary system included within the client node of blood type predictive analysis of the exemplary network of FIG. 1, in accordance with some embodiments.

FIG. 3A is a matrix block diagram associated with the first matrix and row vector, showing generation of the probability for blood types corresponding to a first set of parents in accordance with some embodiments.

FIG. 3B is a matrix block diagram associated with the first matrix in accordance with a second embodiment, showing of the Punnett square table resultant counts corresponding to all combinations for a parent with A⁺ blood types respectively, in accordance with some embodiments.

FIG. 3C is a matrix block diagram associated with the first matrix in accordance with a second embodiment, showing of the Punnett square table resultant counts corresponding to all combinations for a parent with A⁻ blood types respectively, in accordance with some embodiments.

FIG. 3D is a matrix block diagram associated with the first matrix in accordance with a second embodiment, showing of the Punnett square table resultant counts corresponding to all combinations for a parent with B⁺ blood types respectively, in accordance with some embodiments.

FIG. 3E is a matrix block diagram associated with the first matrix in accordance with a second embodiment, showing of the Punnett square table resultant counts corresponding to all combinations for a parent with W blood types respectively, in accordance with some embodiments.

FIG. 3F is a matrix block diagram associated with the first matrix in accordance with a second embodiment, showing of the Punnett square table resultant counts corresponding to all combinations for a parent with AB⁺ blood types respectively, in accordance with some embodiments.

FIG. 3G is a matrix block diagram associated with the first matrix in accordance with a second embodiment, showing of the Punnett square table resultant counts corresponding to all combinations for a parent with O⁺ blood types respectively, in accordance with some embodiments.

FIG. 3H is a matrix block diagram associated with the first matrix in accordance with a second embodiment, showing of the Punnett square table resultant counts corresponding to all combinations for a parent with AB⁻ blood types respectively, in accordance with some embodiments.

FIG. 3I is a matrix block diagram associated with the first matrix in accordance with a second embodiment, showing of the Punnett square table resultant counts corresponding to all combinations for a parent with O⁻ blood types respectively, in accordance with some embodiments.

FIG. 3J is a matrix block diagram associated with the second matrix in accordance with the second embodiment, showing the probability count corresponding to all combinations for parent blood types (A⁺, A⁻ and B⁺), in accordance with some embodiments.

FIG. 3K is a matrix block diagram associated with the second matrix in accordance with the second embodiment, showing the probability count corresponding to all combinations for parent blood types (B−, AB⁺, AB⁻, O⁺, and O⁻), in accordance with some embodiments.

FIG. 4A is an exemplary flow diagram of a method for blood type predictive analysis, in accordance with some embodiments.

FIG. 4B is an exemplary flow diagram of a method for blood type predictive analysis associated with the second embodiment, in accordance with some embodiments.

FIG. 5 is an illustration showing an exemplary computing device, which may implement the embodiments described herein.

### DETAILED DESCRIPTION

The following embodiments describe a blood type predictive analysis system. It can be appreciated by one skilled in the art, that the embodiments may be practiced without some or all of these specific details. In other instances, well known process operations have not been described in detail in order not to unnecessarily obscure the embodiments.

Systems and methods for blood type predictive analysis seem to hold the key for expecting parents in detecting their offspring's blood-type. In particular, systems and methods of predictive analysis can be instrumental when expecting parents want to know their child's blood type. In some embodiments, the system described herein may include a processor coupled to memory, wherein the processor is operable to receive information about each parent's blood type including the dominant-recessive allele pairs. This system can generate a first matrix of a plurality of dominant-recessive allele pairs corresponding to both parents including statistics for all combinations of the dominant-recessive allele pairs. For example, the system can detect the specific number of dominant-recessive allele pairs associated with each parent and assign each to a respective row or column, wherein one parent corresponds to the rows and one parent corresponds to the columns. Next in deriving the first matrix, the system can apply each element of the first matrix to a Punnett square table and detect a count associated with each blood type, whereby each element represents the combined allele pairing for the column and row. Further, the system can generate a row vector specific to both parent's blood type that includes the relative weights of probability associated with each blood type, based upon the first matrix. As output, the system can generate and display a probability rate corresponding to each possible blood type of a child and each associated blood donor matching. Thereby, the systems described herein can enable a parent to detect the most probable blood type that his or her child will have whether from the ABO blood group or the Rh blood group.

In some embodiments, the system may generate the first matrix such that it includes all possible combinations for any parent blood type coupling. In this embodiment, the system can generate a second matrix of all possible parental blood type pairings including the relative weights of probability associated with each blood type. It is from indexing this second matrix that the system can identify a row vector having relative weights associated with each blood type for each parent using the first blood type and second blood type associated with the parents. The system can use these relative weights to generate a probability rate for each corresponding blood type and blood donor.

Advantageously, the systems and methods of predictive analysis described herein can be instrumental to expecting parents seeking knowledge about the future genetic makeup of their offspring by detecting which alleles are dominants or recessives through different combinations with other genes they share as part of this process. A powerful way that people can use these systems and methods may include how medical professionals might predict diseases before symptoms manifest themselves, so that those who suffer can get diagnosed early enough to mitigate or eliminate the disease. Further, a parent's knowledge of the blood type of their child can determine the medication that the child's body can absorb, the strength of their immune system, how quickly the child will age, and possibly the baby's hair color.

In the following description, numerous details are set forth. It will be apparent, however, to one skilled in the art, that the present invention may be practiced without these specific details. In some instances, well-known structures and devices are shown in block diagram form, rather than in detail, in order to avoid obscuring the present invention.

Some portions of the detailed descriptions, which follow, are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, as apparent from the following discussion, it is appreciated that throughout the description, discussions utilizing terms such as “receiving,” “generating, ” “summing, ” “detecting, ” “storing, ” “prompting, ” “assigning,” “applying”, or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The present invention also relates to an apparatus for performing the operations herein. This apparatus may be specially constructed for the required purposes, or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

Reference in the description to “one embodiment” or “an embodiment” means that a particular feature, structure, or characteristic described in connection with the embodiment is included in at least one embodiment of the invention. The phrase “in one embodiment” located in various places in this description does not necessarily refer to the same embodiment. Like reference numbers signify like elements throughout the description of the figures.

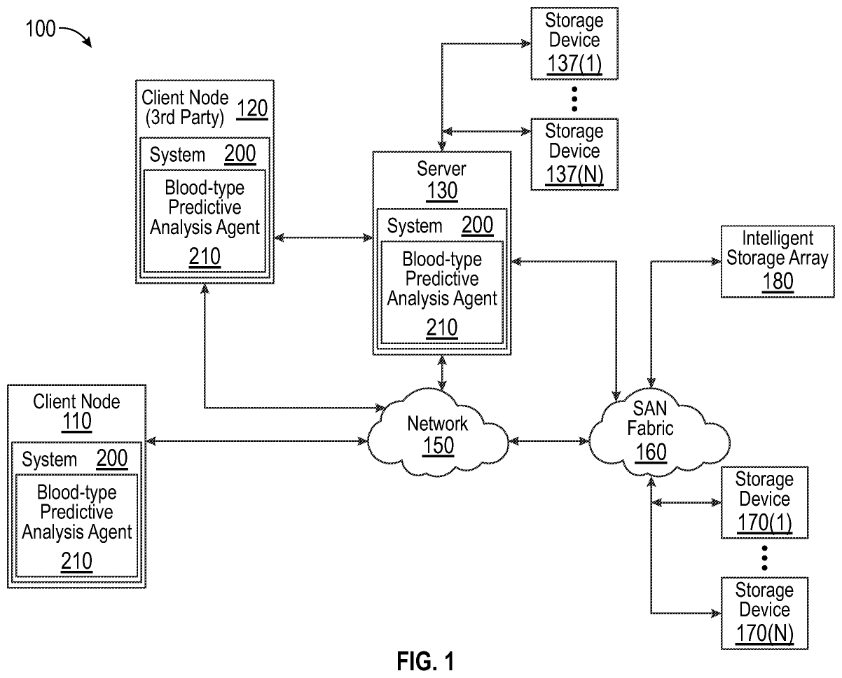

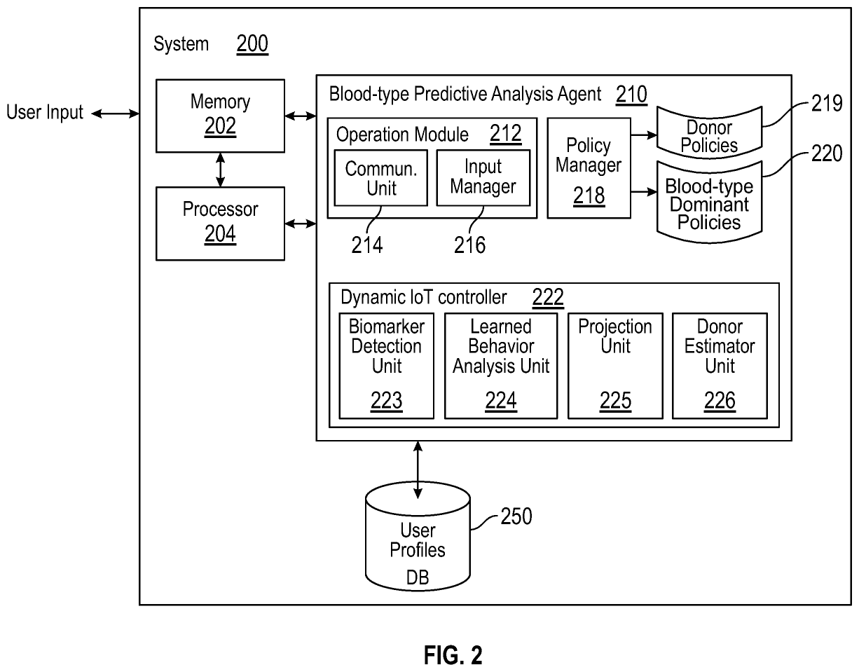

Referring to FIG. 1, the system diagram of an exemplary network incorporating the systems and methods of blood type predictive analysis, in accordance with some embodiments, is shown. The networked system includes at least one client node (110, 120), a network 150, at least one server 130, and a database (not shown in FIG. 1 but shown FIG. 2). As shown in FIG. 1, the exemplary network architecture 100 may include client nodes such as one or more computing devices (110, 120), in direct communication with one another and in communication with the server 130 through network 150. Each client node (110, 120) may possess a blood type predictive analysis agent 210, while each server node (computing device) 130 may also possess a blood type predictive analysis agent 210 (to be described in detail further with reference to FIG. 2). Additionally in some embodiments, third party client nodes 120 may couple to the server node 130 directly or indirectly through network 150 for the purpose of monitoring user blood type predictive analysis settings. For example, when a professional clinician, medical technician, or physician has been granted access to the user's profile, the professional can monitor the output and possibly assert differing policy conditions based upon the medical records of the parents. Additionally, one or more client nodes 145 may be enabled to couple to any one of the client nodes (110, 120) by way of Bluetooth and Wi-Fi communications to access and retrieve data. For example, when both parents are at the doctor's office, a doctor or nurse can directly access the policy rule settings for the blood type predictive analysis on the patient's phone.

In some embodiments, the blood type predictive analysis agent 210 within client node 110, having blood type dominant policies 220 and donor policies 219, may serve as a device that communicates with the server 130 to perform the method of blood type predictive analysis described more in detail below. In other embodiments, the server having the blood type predictive analysis agent 210 may communicate with each client node (110, 120) and serve as the sole agent that performs the method of blood type predictive analysis described herein. The client nodes (110, 120), server 130, and the storage devices (137₁-137_{N}) may reside on the same LAN, or on different LANs that may be coupled together through the Internet, but separated by firewalls, routers, and/or other network devices. In one embodiment, client nodes (110, 120) may couple to network 150 through a mobile communication network. Client nodes (110, 120) may also couple to network 150 through the Plain Old Telephone System (POTS). In another embodiment, the client nodes (110, 120), server 130, and the storage devices (137₁-137_{N}) may reside on different networks. In some embodiments, the server 130 may reside in a cloud network. Although not shown, in various embodiments, the client node (110, 120) may be notebook computers, desktop computers, microprocessor-based or programmable consumer electronics, network appliances, mobile telephones, smart telephones, pagers, radio frequency (RF) devices, infrared (IR) devices, Personal Digital Assistants (PDAs), set-top boxes, cameras, integrated devices combining at least two of the preceding devices, and the like. In some embodiments, the client node (110, 120) may be selected from a group, including a PDA, a Global Positioning System (GPS) device, a monitor, an interactive television, and Internet protocol (IP) phone, a pager, a cellular phone, a satellite phone, and the like.

The server 130 may comprise a processor (not shown), memory (not shown), and blood type predictive analysis 210. In some embodiments, the server 130 may comprise processing software instructions and/or hardware logic required for blood type predictive analysis and blood donor matching according to the embodiments described herein. The server 130 may provide remote cloud storage capabilities for user profiles, blood type predictions, donor database, and various types of dominant blood type and donor policies associated, through the storage device 190 coupled by network 180. Further, server 130 may couple to one or more tape-out devices (not shown) or any other secondary datastore. As such, a database of user profile data and blood type and/or donor policies may be stored within a local data store, remote disks, secondary data storage devices, or tape-outs devices (not shown). In some embodiments, the client nodes (110, 120) may retrieve previous results relating to the user's profile and dominant blood type and donor predictions relating to user input(s) from a remote datastore to a local data store (not shown). In other embodiments, a database of dominant blood type and donor policies, prior blood type and donor predictions, along with medical history data, and the like may be stored locally on the client nodes 110, 120, or the server 130. In particular, for remote storage purposes, the local data storage unit (not shown) can be one or more centralized data repositories having mappings of respective associations between each fragment data and its location within remote storage devices. The local data store may represent a single or multiple data structures (databases, repositories, files, etc.) residing on one or more mass storage devices, such as magnetic or optical storage based disks, tapes or hard drives. This local data store may be an internal component of the server 130. In the alternative, the local data store 137 (1-N) also may couple externally to server 130 as shown in FIG. 1, or remotely through a network 160 (storage devices 170(1-N)). Further, the server 130 may communicate with the remote storage devices over a public or private network. Although not shown, in various embodiments, the server 130 may be a notebook computer, desktop computer, microprocessor-based or programmable consumer electronics, network appliance, mobile telephone, smart telephone, radio frequency (RF) device, infrared (IR) device, Personal Digital Assistant (PDA), set-top box, an integrated device combining at least two of the preceding devices, and the like.

In some embodiments, one or more storage devices 137(1)-(N) may be directly attached to server 130. Storage devices 137(1)-(N) generally represent any type or form of storage device or medium capable of storing data and/or other computer-readable instructions. In certain embodiments, storage devices 137(1)-(N) may represent Network-Attached Storage (NAS) devices configured to communicate with server 130 using various protocols, such as Network File System (NFS), Server Message Block (SMB), or Common Internet File System (CIFS).

Server 130 may also be connected to a Storage Area Network (SAN) fabric 160. SAN fabric 160 generally represents any type or form of computer network or architecture capable of facilitating communication between a plurality of storage devices. SAN fabric 160 may facilitate communication between server 130 and a plurality of storage devices 170(1)-(N) and/or an intelligent storage array 180. SAN fabric 160 may also facilitate, via network 150 and server 130, communication between client systems (110, 120), and storage devices 170(1)-(N) and/or intelligent storage array 180 in such a manner that devices 170(1)-(N) and array 180 appear as locally attached devices to client systems (110, 120).

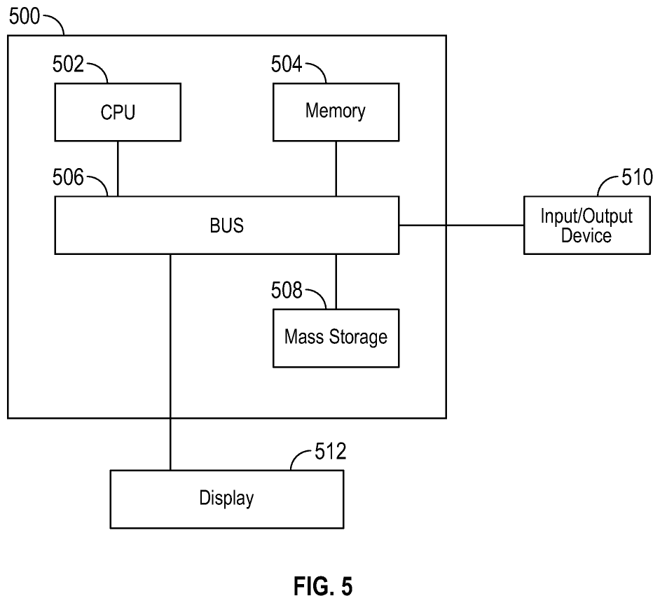

In certain embodiments, and with reference to exemplary computing system 500 of FIG. 5, a communication interface may be used to provide connectivity between each client system (110, 120) and network 150. Client systems (110, 120) may be able to access information on server 130 using, for example, a web browser or other client software. Such software may allow client systems (110, 120) to access data hosted by server 130, storage devices 137(1)-(N), storage devices 170(1)-(N), or intelligent storage array 180. Although FIG. 1 depicts the use of a network (such as the Internet) for exchanging data, the embodiments described and/or illustrated herein are not limited to the Internet or any particular network-based environment.

In at least one embodiment, all or a portion of one or more of the exemplary embodiments disclosed herein may be encoded as a computer program and loaded onto and executed by server 130, storage devices 137(1)-(N), storage devices 170(1)-(N), or intelligent storage array 180, or any combination thereof. All or a portion of one or more of the exemplary embodiments disclosed herein may also be encoded as a computer program, stored in server 130, and distributed to client systems (110, 120) over network 150.

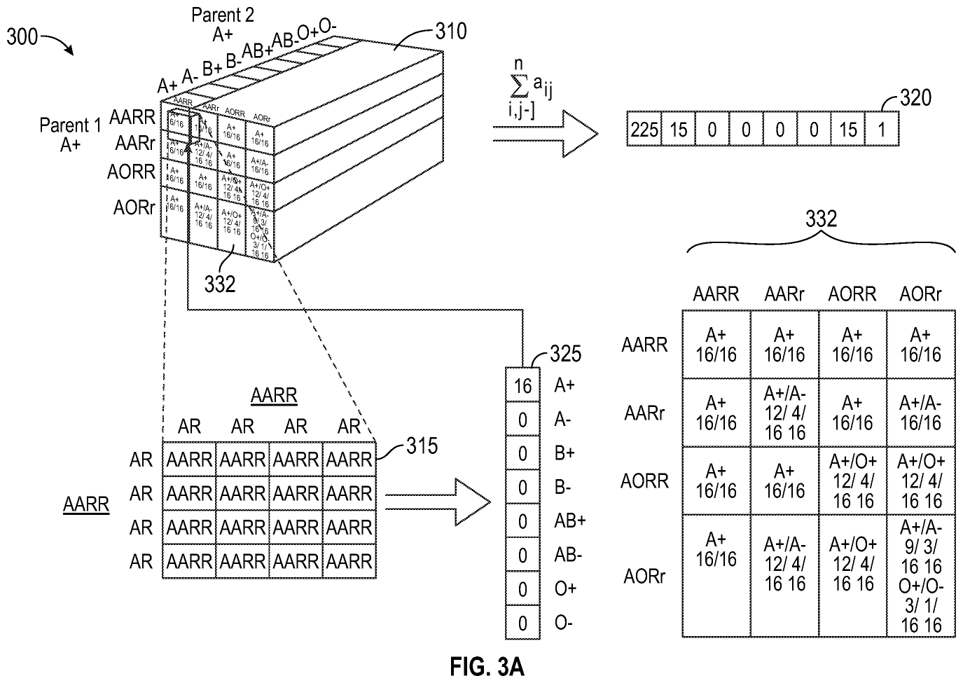

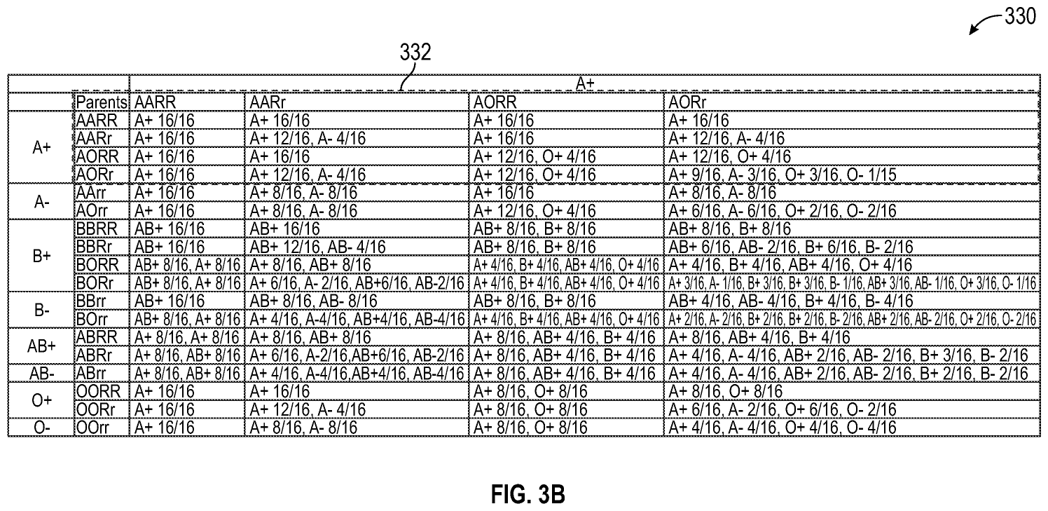

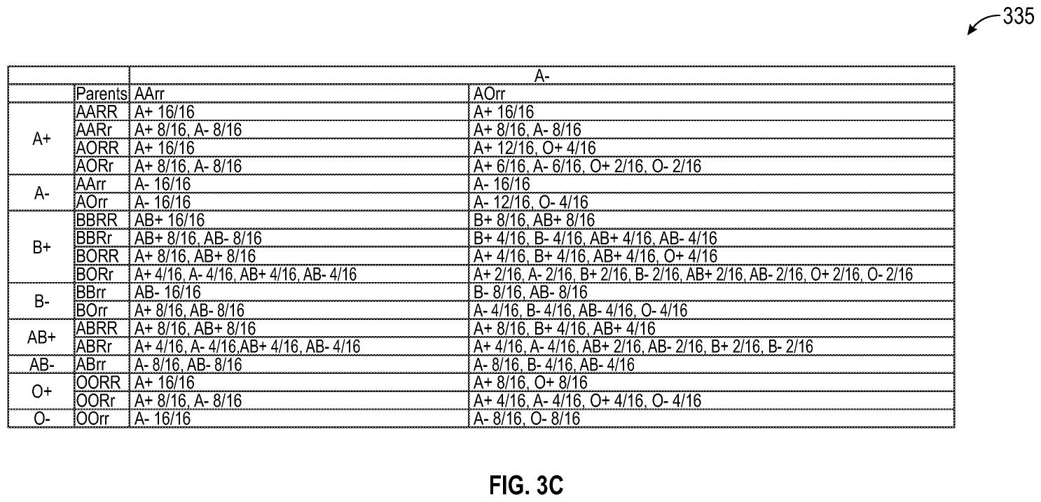

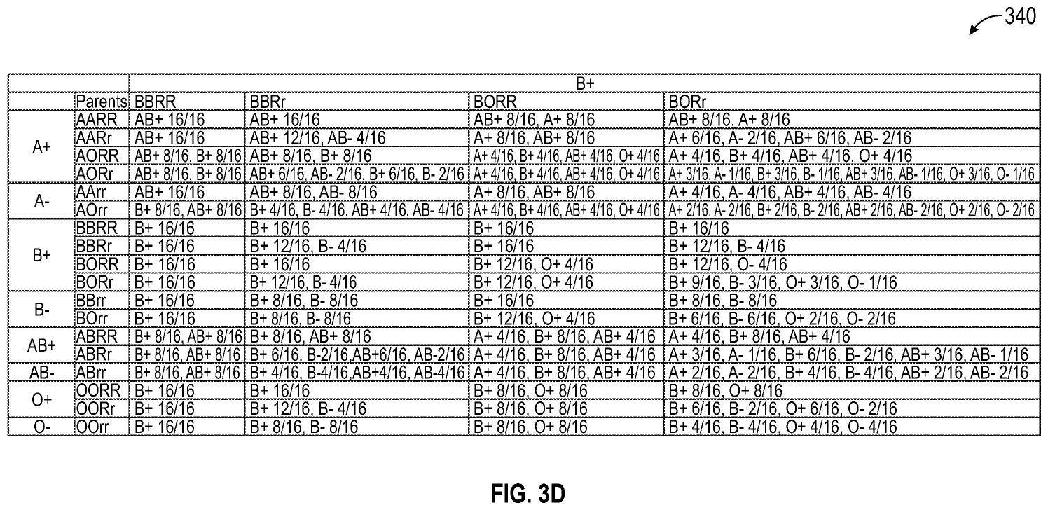

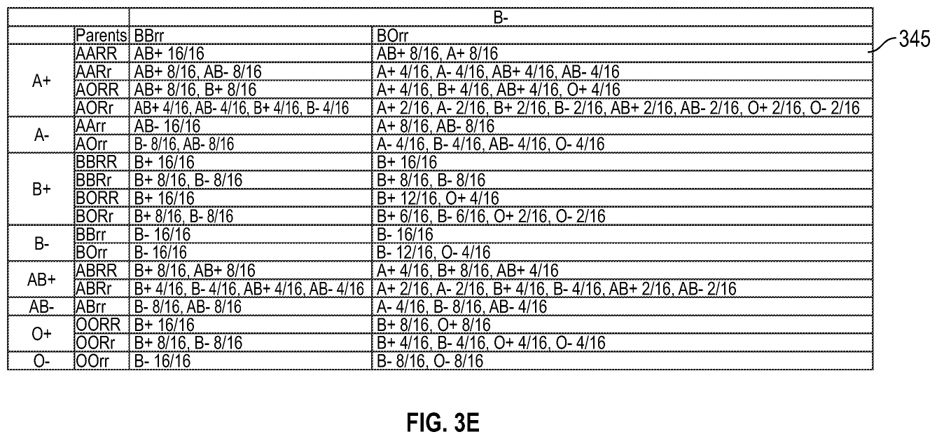

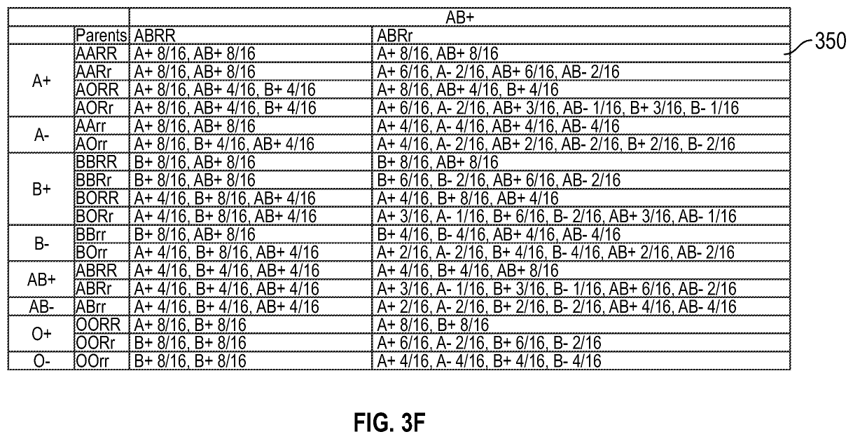

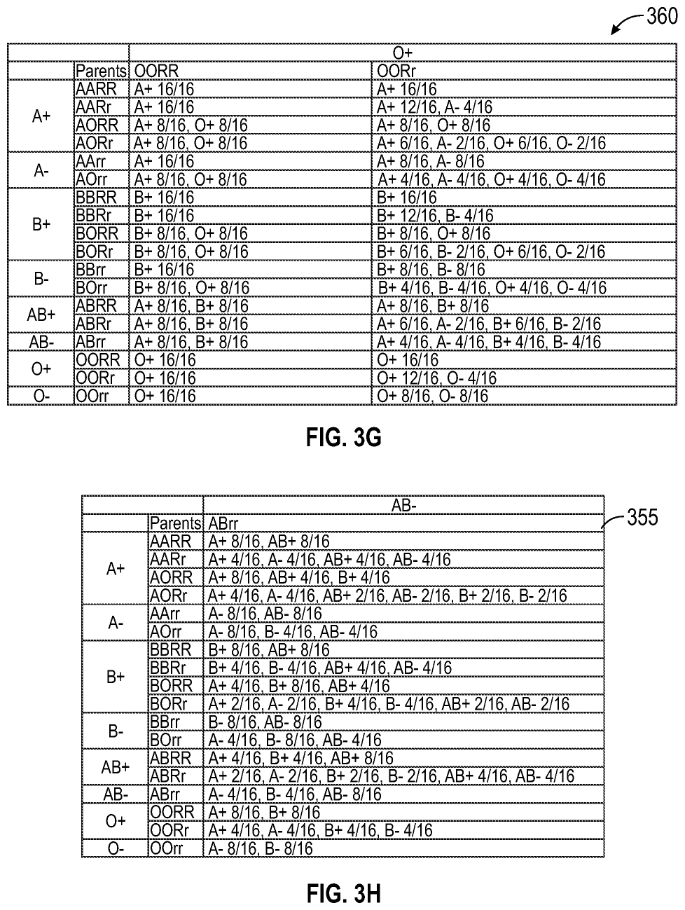

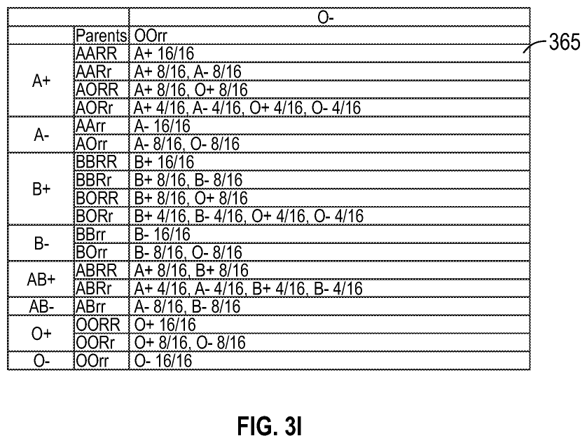

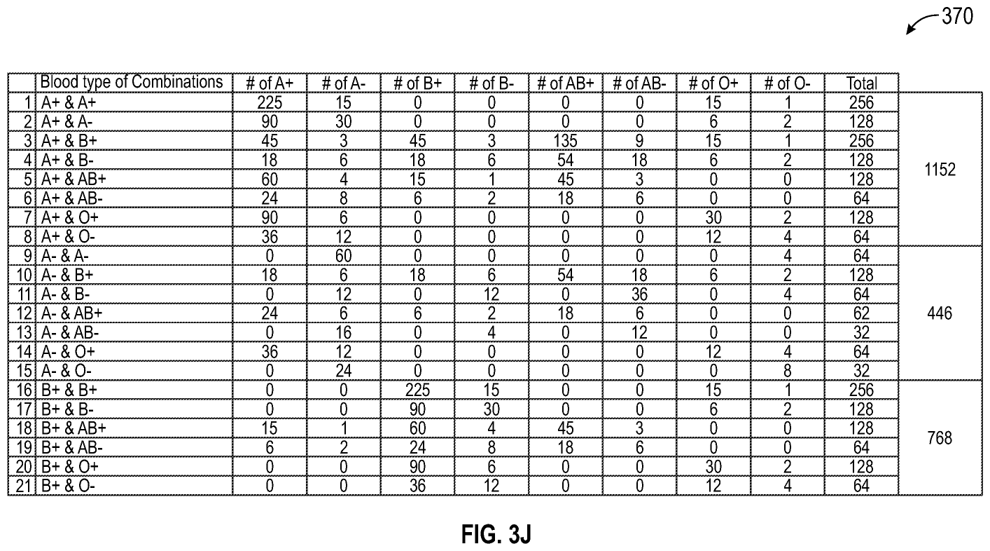

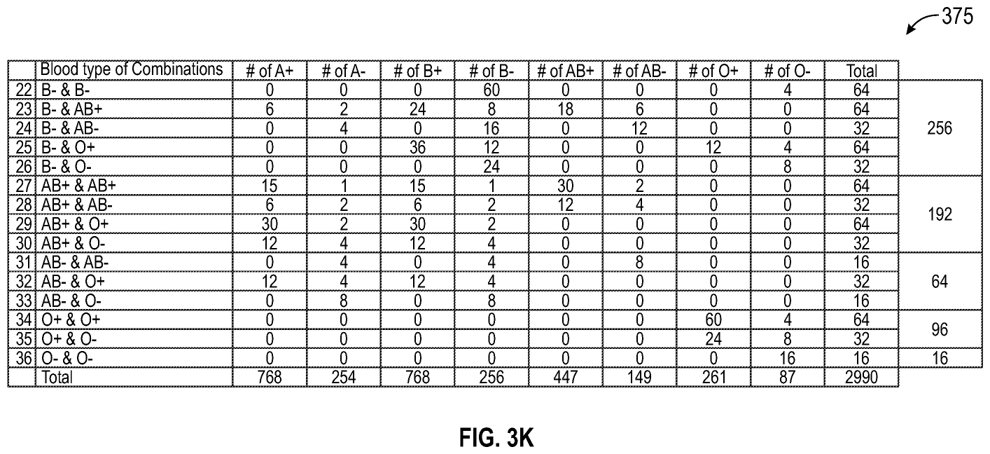

In operation, the blood type predictive analysis agent 210 within system 200 of client 110 can prompt the user for input associated with a first and a second parent, wherein the first blood type and second blood type each includes a dominant-recessive allele pair. Next, the predictive analysis agent 210 may generate a first matrix of a plurality of dominant-recessive allele pairs corresponding to the first parent and the second parent. For example, system 200 can detect the specific number of dominant-recessive allele pairs associated with each parent and assign each to a respective row or column of matrix (to be described in more detail with respect to FIGS. 2 and 3A), wherein one parent is associated with each row and one parent is associated with each column. Next in deriving the first matrix, system 200 can apply each element of the first matrix to a Punnett square table and detect a count associated with each blood type, whereby each element represents the combined allele pairing for the corresponding column and row. The system 200 can further generate a row vector, having relative weights associated with each blood type, based upon the first matrix. For example, a count of dominant allele(s) can be detected and assigned to an element of a row vector associated with each blood type, such as A⁺, A⁻, B⁺, B⁻, AB⁺, AB⁻, O⁺, O⁻, and the like. Additionally, the system 200 may sum the row vector to generate a total value, wherein each element of the row vector can be divided by the total value to generate a resultant for each blood type. Moreover, the system 200 can multiply each resultant by 100 to generate a probability rate corresponding to each blood type. Furthermore, the system 200 can detect the blood donor matching for each blood type. As output, the system 200 can display the probability rate corresponding to each possible blood type of a child. Further, system 200 can display the associated blood donor matching.

In some embodiments, the blood type predictive analysis agent 210 within system 200 of client 110 can generate the first matrix such that it includes all possible combinations for any parent blood type coupling prior to user input of parent blood type data. For example, the first matrix may include all of the combinations associated with each blood type, such as A⁺, A⁻, B⁺, B⁻, AB⁺, AB⁻, O⁺, O⁻, and the like (to be described in more detail with respect to FIGS. 3B-3I). In this embodiment, blood type predictive analysis agent 210 may generate a second matrix of all possible parental blood type pairings including the relative weights of probability associated with each blood type based upon the first matrix. For example, the second matrix may include the associated probability counts for all of the combinations associated with each blood type, such as A⁺, A⁻, B⁺, B⁻, AB⁺, AB⁻, O⁺, O⁻, and the like (to be discussed more in detail with reference to FIGS. 3J and 3K). Next, the blood type predictive analysis system 200 may prompt the user for input of a first blood type associated with a first parent and a second blood type associated with a second parent, wherein the first blood type and second blood type each includes a dominant-recessive allele pair. The blood type predictive analysis agent 210 may further index the second matrix using the associated first blood type and second blood type corresponding to each parent to identify a row vector having relative weights associated with each blood type that specifically corresponds to the parents. These relative weights may be used to generate a probability rate for each corresponding blood type and blood donor. In particular, blood type predictive analysis agent 210 may sum the row vector to generate a total value, wherein each element of the row vector can be divided by the total value to generate a resultant for each blood type. Moreover, blood type predictive analysis agent 210 can multiply each resultant by 100 to generate a probability rate for each corresponding blood type. Furthermore, blood type predictive analysis agent 210 may detect the blood donor matching for each blood type. As output, system 200 of client 110 may display the probability rate corresponding to each possible blood type of a child and associated blood donor matching in order of ranking.

It is appreciated that the components of an exemplary operating environment 100 are exemplary and more or fewer components may be present in various configurations. It is appreciated that the operating environment may be part of a distributed computing environment, a cloud computing environment, a client server environment, and the like.

Referring now to FIG. 2, a block diagram of an exemplary system included within the client node of blood type predictive analysis of the exemplary network of FIG. 1, in accordance with some embodiments is presented. Exemplary system 200 may be implemented in a variety of ways. For example, all or a portion of exemplary system 200 may represent portions of exemplary network 100 in FIG. 1. As illustrated in FIG. 1, exemplary system 200 may include a memory 202, a processor 204, and a blood type predictive analysis agent 210 having a storage database 250 for storing user profile information. For example, and as will be explained in greater detail below, blood type predictive analysis agent 210 may include an operations module 212; a policy manager 218 with associated donor policies 219 and blood type dominant policies 220; and a predictive unit 222. The operations module 212 can include a communications unit 214 for generating communication signals to receive and transport user profile data and policy rule data. The operations module 212 can further include an input manager 216 for receiving user input regarding the first and second blood type data associated with the first and second parents respectively. The policy manager 218 in collaboration with predictive unit 22 may select blood type dominant rules from the blood type dominant policies 220. Further, the policy manager 218 may select donor rules from the donor policies 219 for the purpose of detecting a donor match in alignment with the matrix or vector containing probability information (to be described in detail below).

In some embodiments, the predictive unit 222 may include a Punnett square table unit 223, a matrix assignment unit 224, a probability generator 225, and a donor estimator unit 226. The Punnett square table unit 223 can generate the probable count associated with each blood type for each allele pairing. For example, given the allele pairing AARR and AARR, the Punnett square table unit 223 can generate the associated count of probability for each blood type, such as A⁺, A⁻, B⁺, B⁻, AB⁺, AB⁻, O⁺, O⁻, and the like. The matrix assignment unit 224 can generate a first matrix having the relative probability counts based upon the output of the Punnett square table unit 223. The probability generator 225 can generate the probability for each particular blood type based upon the first matrix. The donor estimator unit 226 can detect and assign a donor matching probability that corresponds with the output of the probability generator 225.

In operation, the processor 204 in collaboration with the operation module 212 may retrieve the user profile from a local storage unit 250. Further, the processor 204 in collaboration with the input manager 216 may prompt the user for input associated with a first and a second parent, wherein the first blood type and second blood type each includes a dominant-recessive allele pair. Next, the matrix assignment unit 224 of the predictive unit 222 may generate a first matrix of a plurality of dominant-recessive allele pairs corresponding to the first parent and the second parent. For example, the predictive unit 222 can detect the specific number of dominant-recessive allele pairs associated with each parent and assign each to a respective row or column of matrix (to be described in more detail with respect to FIG. 3A), wherein one parent is associated with each row and one parent is associated with each column. Next in deriving the first matrix, the Punnett square table unit 223 can apply each element of the first matrix to a Punnett square table and detect a count associated with each blood type, whereby each element represents the combined allele pairing for the corresponding column and row. The matrix assignment unit 224 can further generate a row vector, having relative weights associated with each blood type, based upon the first matrix. For example, a count of dominant allele(s) can be detected and assigned to an element of a row vector associated with each blood type, such as A⁺, A⁻, B⁺, B⁻, AB⁺, AB⁻, O⁺, O⁻, and the like. Additionally, the probability generator 225 may sum the row vector to generate a total value, wherein each element of the row vector can be divided by the total value to generate a resultant for each blood type. Moreover, the probability generator 225 can multiply each resultant by 100 to generate a probability rate corresponding to each blood type. Furthermore, the donor estimator unit 226 can detect the blood donor matching for each blood type. As output, the processor 204 in collaboration with the input manager 216 can display the probability rate corresponding to each possible blood type of a child. Further, the processor 204 in collaboration with the input manager 216 may display the associated blood donor matching.

Referring now to FIG. 3A, a matrix block diagram associated with the first matrix and row vector, showing generation of the probability for blood types corresponding to a first set of parents in accordance with some embodiments is shown. In particular, blood type predictive analysis system 200 may generate a first matrix 310 of a plurality of dominant-recessive allele pairs corresponding to the first parent and the second parent. For example, system 200 can detect the specific number of dominant-recessive allele pairs associated with each parent and assign each to a respective row or column of matrix 310. More particularly as shown, one parent corresponds to the rows (Parent 1) and another parent corresponds to the columns (Parent 2). Next in deriving the first matrix, the system can apply each element of the first matrix to a Punnett square table 315 and detect a count associated with each blood type (as shown in matrix 332), whereby each element represents the combined allele pairing for the corresponding column and row, as is shown in vector 325. The blood type predictive analysis system 200 may further generate a row vector 320, having relative weights associated with each blood type, based upon the first matrix 310. For example, a count of dominant allele(s) can be detected and assigned to an element of a row vector 320 associated with each blood type, such as A⁺, A⁻, B⁺, B⁻, AB⁺, AB⁻, O⁺, O⁻, and the like. Additionally, blood type predictive analysis system 200 may sum the elements of row vector 320 to generate a total value, wherein each element of the row vector can be divided by the total value to generate a resultant for each blood type. Accordingly, blood type predictive analysis system 200 may multiply each resultant by 100 to derive the probability rate corresponding to each blood type.

Referring now to FIGS. 3B and 3C, a matrix block diagram associated with the first matrix in accordance with a second embodiment, showing of the Punnett square table resultant counts corresponding to all combinations for a parent with blood types A⁺, A⁻, B⁺, B⁻, AB⁺, AB⁻, O⁺, O⁻, and the like respectively, in accordance with some embodiments is shown. In particular, blood type predictive analysis system 200 can generate the first matrix such that it includes all possible combinations for any parent blood type coupling. For example, the first matrix may include all of the combinations associated with each blood type, such as A⁺ (330), A⁻ (335), B⁺ (340), B⁻ (345), AB⁺ (350), AB⁻ (355), O⁺ (360), O⁻ (365), and the like (FIGS. 3B and 3C). In this embodiment, blood type predictive analysis system 200 can generate a second matrix (370, 375) of all possible parental blood type pairings including the relative weights of probability associated with each blood type based upon the first matrix (FIGS. 3J and 3K). For example, the second matrix may include the associated probability counts for all of the combinations associated with each blood type, such as A⁺, A⁻, B⁺, B⁻, AB⁺, AB⁻, O⁺, O⁻, and the like (FIGS. 3J and 3K, 370 and 375). Next, the blood type predictive analysis system 200 can prompt the user and receive user input of a first blood type associated with a first parent and receiving user input of a second blood type associated with a second parent, wherein the first blood type and second blood type each includes a dominant-recessive allele pair. Next, the blood type predictive analysis system 200 may index the second matrix (370, 375) using the associated first blood type and second blood type corresponding to each parent to identify a row vector having relative weights associated with each blood type that specifically corresponds to the parents as a pair. These relative weights can be used to generate a probability rate for each corresponding blood type and blood donor. In particular, the method may include summing the elements of row within the second matrix that correspond to the parents blood type pairing in an effort to generate a total value. System 200 can divide each element of the row by the total value to generate a resultant for each blood type. Moreover, blood type predictive analysis agent can multiply each resultant by 100 to generate a probability rate for each corresponding blood type associated with the child of the parents. The agent 210 can rank the probability rates and display the most probable result to the user in some embodiments. Furthermore, agent 210 can detect the blood donor matching for each blood type. As output, the agent 210 can display a probability rate corresponding to each possible blood type of a child, along with an associated blood donor matching.

As used herein, the term agent might describe a given unit of functionality that can be performed in accordance with one or more embodiments of the present invention. As used herein, an agent might be implemented utilizing any form of hardware, software, or a combination thereof. For example, one or more processors, controllers, ASICs, PLAs, PALs, CPLDs, FPGAs, logical components, software routines or other mechanisms might be implemented to make up an agent. In implementation, the various agents described herein might be implemented as discrete agents or the functions and features described can be shared in part or in total among one or more agents. In other words, as would be apparent to one of ordinary skill in the art after reading this description, the various features and functionality described herein may be implemented in any given application and can be implemented in one or more separate or shared agents in various combinations and permutations. Even though various features or elements of functionality may be individually described or claimed as separate agents, one of ordinary skill in the art will understand that these features and functionality can be shared among one or more common software and hardware elements, and such description shall not require or imply that separate hardware or software components are used to implement such features or functionality.

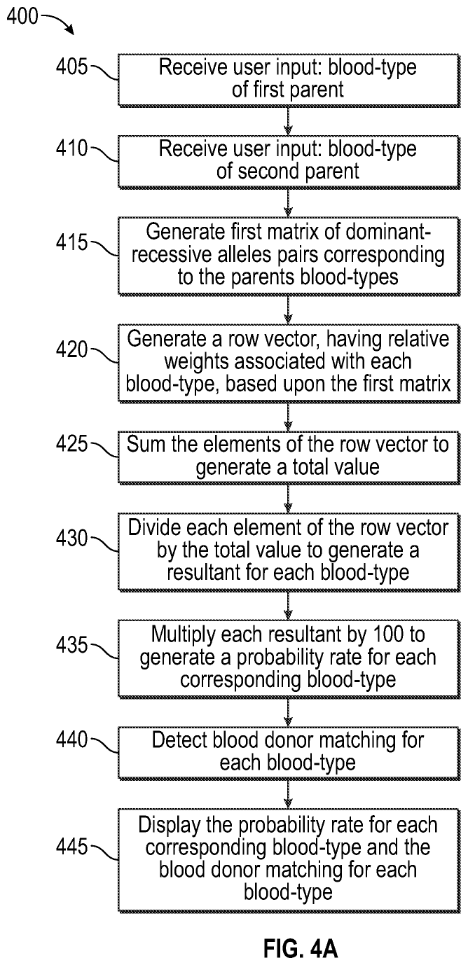

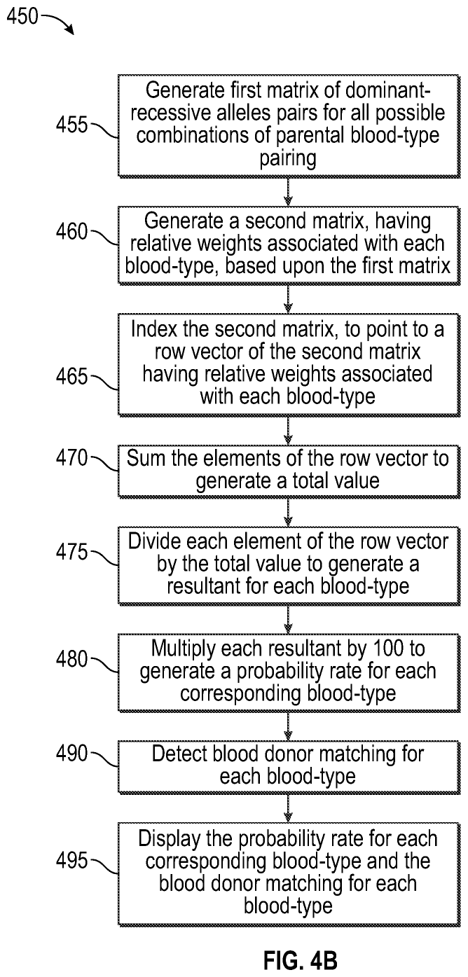

Referring now to FIG. 4A, an exemplary flow diagram of a method of blood type predictive analysis in accordance with some embodiments is illustrated. As shown, in respective actions 405 and 410, the blood type predictive analysis system 200 may include a method of receiving user input of a first blood type associated with a first parent and receiving user input of a second blood type associated with a second parent, wherein the first blood type and second blood type each includes a dominant-recessive allele pair. Next in an action 415, the method may include generating a first matrix of a plurality of dominant-recessive allele pairs corresponding to the first parent and the second parent. For example, system 200 can detect the specific number of dominant-recessive allele pairs associated with each parent and assign each to a respective row or column of matrix 310 (FIG. 3A), wherein one parent corresponds to the rows (Parent 1) and one parent corresponds to the columns (Parent 2). Next in deriving the first matrix, the system can apply each element of the first matrix to a Punnett square table (315) and detect a count associated with each blood type, whereby each element represents the combined allele pairing for the corresponding column and row, and stored in a register/vector (325). The method in an action 420 may further include generating a row vector, having relative weights associated with each blood type, based upon the first matrix. For example, a count of dominant allele(s) can be detected and assigned to an element of a row vector 320 associated with each blood type, such as A⁺, A⁻, B⁺, B⁻, AB⁺, AB⁻, O⁺, O⁻, and the like (FIG. 3A). Additionally, the method may include summing the row vector to generate a total value (in an action 425), wherein each element of the row vector can be divided by the total value to generate a resultant for each blood type (in an action 430). Moreover, the method may include multiplying each resultant by 100 to generate a probability rate corresponding to each blood type (in an action 435). Furthermore in an action 440, the method may include detecting the blood donor matching for each blood type. As output, the method may include generating and displaying a probability rate corresponding to each possible blood type of a child and associated blood donor matching in an action 445.

In some embodiments as is shown in FIG. 4B, the method may include generating the first matrix such that it includes all possible combinations for any parent blood type coupling (in an action 455). For example, the first matrix may include all of the combinations associated with each blood type, such as A⁺ (330), A⁻ (335), B⁺ (340), B⁻ (345), AB⁺ (350), AB⁻ (355), O⁺ (360), O⁻ (365), and the like (FIGS. 3B and 3C). In this embodiment, the method may include generating a second matrix of all possible parental blood type pairings including the relative weights of probability associated with each blood type based upon the first matrix (in an action 460). For example, the second matrix may include the associated probability counts for all of the combinations associated with each blood type, such as A⁺, A⁻, B⁺, B⁻, AB⁺, AB⁻, O⁺, O⁻, and the like (FIG. 3D, 370). In respective actions 465 and 470, the blood type predictive analysis system 200 may include a method of receiving user input of a first blood type associated with a first parent and receiving user input of a second blood type associated with a second parent, wherein the first blood type and second blood type each includes a dominant-recessive allele pair. The method may further include indexing the second matrix using the associated first blood type and second blood type corresponding to each parent to identify a row vector having relative weights associated with each blood type that specifically corresponds to the parents, in an action 465. These relative weights may be used to generate a probability rate for each corresponding blood type and blood donor. In particular, the method may include summing the row vector to generate a total value (in an action 470), wherein each element of the row vector can be divided by the total value to generate a resultant for each blood type (in an action 475). Moreover, the method may include multiplying each resultant by 100 to generate a probability rate for each corresponding blood type (in an action 480). Furthermore in an action 490, the method may include detecting the blood donor matching for each blood type. As output, the method may include generating and displaying a probability rate corresponding to each possible blood type of a child and associated blood donor matching in an action 495.

It should be appreciated that the methods described herein may be performed with a digital processing system, such as a conventional, general-purpose computer system. Special purpose computers, which are designed or programmed to perform only one function may be used in the alternative. FIG. 5 is an illustration showing an exemplary computing device, which may implement the embodiments described herein. The computing device of FIG. 5 may be used to perform embodiments of the functionality for performing the blood type predictive analysis in accordance with some embodiments. The computing device includes a central processing unit (CPU) 502, which is coupled through a bus 506 to a memory 504, and mass storage device 508. Mass storage device 508 represents a persistent data storage device such as a floppy disc drive or a fixed disc drive, which may be local or remote in some embodiments. The mass storage device 508 could implement a backup storage, in some embodiments. Memory 504 may include read only memory, random access memory, etc. Applications resident on the computing device may be stored on or accessed through a computer readable medium such as memory 504 or mass storage device 508 in some embodiments. Applications may also be in the form of modulated electronic signals accessed through a network modem or other network interface of the computing device. It should be appreciated that CPU 502 may be embodied in a general-purpose processor, a special purpose processor, or a specially programmed logic device in some embodiments.

Display 512 is in communication with CPU 502, memory 504, and mass storage device 508, through bus 506. Display 512 is configured to display any visualization tools or reports associated with the system described herein. Input/output device 510 is coupled to bus 506 in order to communicate information in command selections to CPU 502. It should be appreciated that data to and from external devices may be communicated through the input/output device 510. CPU 502 can be defined to execute the functionality described herein to enable the functionality described with reference to FIGS. 1-4B. The code embodying this functionality may be stored within memory 504 or mass storage device 508 for execution by a processor such as CPU 502 in some embodiments. The operating system on the computing device may be iOS™, MS-WINDOWS™, OS/2™, UNIX™, LINUX™, or other known operating systems. It should be appreciated that the embodiments described herein may be integrated with virtualized computing systems also.

The embodiments can also be embodied as computer readable code on a non-transitory computer readable medium. The computer readable medium is any data storage device that can store data, which can be thereafter read by a computer system. Examples of the computer readable medium include hard drives, network attached storage (NAS), read-only memory, random-access memory, CD-ROMs, CD-Rs, CD-RWs, magnetic tapes, flash memory devices, and other optical and non-optical data storage devices. The computer readable medium can also be distributed over a network coupled computer system so that the computer readable code is stored and executed in a distributed fashion. Embodiments described herein may be practiced with various computer system configurations including hand-held devices, tablets, microprocessor systems, microprocessor-based or programmable consumer electronics, minicomputers, mainframe computers and the like. The embodiments can also be practiced in distributed computing environments where tasks are performed by remote processing devices that are linked through a wire-based or wireless network.

In various embodiments, one or more portions of the methods and mechanisms described herein may form part of a cloud-computing environment. In such embodiments, resources may be provided over the Internet as services according to one or more various models. Such models may include Infrastructure as a Service (IaaS), Platform as a Service (PaaS), and Software as a Service (SaaS). In IaaS, computer infrastructure is delivered as a service. In such a case, the computing equipment is generally owned and operated by the service provider. In the PaaS model, software tools and underlying equipment used by developers to develop software solutions may be provided as a service and hosted by the service provider. SaaS typically includes a service provider licensing software as a service on demand. The service provider may host the software, or may deploy the software to a customer for a given period of time. Numerous combinations of the above models are possible and are contemplated.

In the above description, numerous details are set forth. It will be apparent, however, to one skilled in the art, that the present invention may be practiced without these specific details. In some instances, well-known structures and devices are shown in block diagram form, rather than in detail, in order to avoid obscuring the present invention.

It is to be understood that the above description is intended to be illustrative, and not restrictive. Many other embodiments will be apparent to those of skill in the art upon reading and understanding the above description. Although the present invention has been described with reference to specific exemplary embodiments, it will be recognized that the invention is not limited to the embodiments described, but can be practiced with modification and alteration within the spirit and scope of the appended claims. Specific functional details disclosed herein are merely representative for purposes of describing embodiments. Embodiments may, however, be embodied in many alternate forms and should not be construed as limited to only the embodiments set forth herein. Accordingly, the specification and drawings are to be regarded in an illustrative sense rather than a restrictive sense. The scope of the invention should, therefore, be determined with reference to the appended claims, along with the full scope of equivalents to which such claims are entitled.

Although the method operations were described in a specific order, it should be understood that other operations may be performed in between described operations, described operations may be adjusted so that they occur at slightly different times or the described operations may be distributed in a system which allows the occurrence of the processing operations at various intervals associated with the processing.

It should be understood that although the terms first, second, etc. may be used herein to describe various steps or calculations, these steps or calculations should not be limited by these terms. These terms are only used to distinguish one step or calculation from another. For example, a first calculation could be termed a second calculation, and, similarly, a second step could be termed a first step, without departing from the scope of this disclosure. As used herein, the term “and/or” and the “I” symbol includes any and all combinations of one or more of the associated listed items. As used herein, the singular forms “a”, “an” and “the” are intended to include the plural forms as well, unless the context clearly indicates otherwise. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. Therefore, the terminology used herein is for the purpose of describing particular embodiments only and is not intended to be limiting.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved. With the above embodiments in mind, it should be understood that the embodiments might employ various computer-implemented operations involving data stored in computer systems. These operations are those requiring physical manipulation of physical quantities. Usually, though not necessarily, these quantities take the form of electrical or magnetic signals capable of being stored, transferred, combined, compared, and otherwise manipulated. Further, the manipulations performed are often referred to in terms, such as producing, identifying, determining, or comparing. Any of the operations described herein that form part of the embodiments are useful machine operations. The embodiments also relate to a device or an apparatus for performing these operations. The apparatus can be specially constructed for the required purpose, or the apparatus can be a general-purpose computer selectively activated or configured by a computer program stored in the computer. In particular, various general-purpose machines can be used with computer programs written in accordance with the teachings herein, or it may be more convenient to construct a more specialized apparatus to perform the required operations.

A module, an application, a layer, an agent or other method-operable entity could be implemented as hardware, firmware, or a processor executing software, or combinations thereof. It should be appreciated that, where a software-based embodiment is disclosed herein, the software can be embodied in a physical machine such as a controller. For example, a controller could include a first module and a second module. A controller could be configured to perform various actions, e.g., of a method, an application, a layer or an agent.

What is claimed is:

1. A method of blood type predictive analysis for an offspring, comprising:
receiving user input of a first blood type associated with a first parent;
receiving user input of a second blood type associated with a second parent, wherein the first blood type and second blood type each includes a dominant-recessive allele pair;
generating a first matrix of a plurality of dominant-recessive alleles pairs corresponding to the first parent and the second parent;
generating a row vector, having relative weights associated with each blood type, based upon the first matrix;
summing the row vector to generate a total value;
dividing each element of the row vector by the total value to generate a resultant for each blood type; and
multiplying each resultant by 100 to generate a probability rate for each corresponding blood type.

2. The method of claim 1, wherein receiving the user input comprises,
prompting the user for input associated with the first parent relating to a first blood type having a dominant-recessive allele pair;
prompting the user for input associated with the second parent relating to a first blood type having a dominant-recessive allele pair; and
storing the input in a register.

3. The method of claim 1, wherein generating of the first matrix comprises,
detecting a number J of dominant-recessive allele pairs associated with the first parent;
detecting a number K of dominant-recessive allele pair associated with the second parent;
assigning each of the J of dominant-recessive allele pairs with a column of the first matrix;
assigning each of the K of dominant-recessive allele pairs with a row of the first matrix;
applying each of the J and K dominant-recessive pairs to a Punnett square table;
detecting a count for each possible blood type based upon blood type policies relating to dominant and recessive traits; and
storing the count at the intersection of the J column and K row of the first matrix.

4. The method of claim 3, wherein applying each of the J and K dominant-recessive pairs to the Punnett square table comprises,
selecting iteratively each dominant-recessive pair associated with the first parent from 1 to J;
storing each dominant-recessive pair in a first register associated with a column of the matrix;
selecting iteratively each dominant-recessive pair associated with the second parent from form 1 to K;
storing each dominant-recessive pair in a second register associated with a row of the matrix;
retrieving iteratively from the first and second registers a set of dominant-recessive pairs corresponding to each intersection of the matrix; and
asserting various combinations of the set of dominant-recessive pairs from each intersection of the Punnett square table.

5. The method of claim 1, wherein generating of the row vector comprises,
detecting a sum of squares from the Punnett square table associated with each blood type;
setting each sum to be the relative weight associated with each blood type; and
assigning each relative weight to an associated one of the elements of the row vector.

6. The method of claim 1, further comprising:
retrieving elements from an associated blood donor matrix;
applying each element to the first matrix;
detecting blood donor matching for each blood type; and
displaying the probability rate for each corresponding blood type and the blood donor matching for each blood type.

7. The method of claim 1, wherein the first blood type and second blood type each are associated with blood group systems of ABO and Rh.

8. A blood type predictive analysis system comprising:
a memory; and
a processor operable to:
receive user input of a first blood type associated with a first parent;
receive user input of a second blood type associated with a second parent, wherein the first blood type and second blood type each includes a dominant-recessive allele pair and each are associated with blood group systems of ABO and Rh;
generate a first matrix of a plurality of dominant-recessive alleles pairs corresponding to the first parent and the second parent;
generate a row vector, having relative weights associated with each blood type, based upon the first matrix;
sum the row vector to generate a total value;
divide each element of the row vector by the total value to generate a resultant for each blood type; and
multiply each resultant by 100 to generate a probability rate for each corresponding blood type.

9. The blood type predictive analysis system of claim 8, wherein the processor, for receiving the user input, operable to:
prompt the user for input associated with the first parent relating to a first blood type having a dominant-recessive allele pair;
prompt the user for input associated with the second parent relating to a first blood type having a dominant-recessive allele pair; and
store the input in a register.

10. The blood type predictive analysis system of claim 8, wherein the processor, for generating of the matrix, operable to:
detect a number J of dominant-recessive allele pairs associated with the first parent;
detect a number K of dominant-recessive allele pair associated with the second parent;
assign each of the J of dominant-recessive allele pairs with a column of the matrix;
assign each of the K of dominant-recessive allele pairs with a row of the matrix;
apply each of the J and K dominant-recessive pairs to a Punnett square table;
detect a count for each possible blood type based upon blood type policies relating to dominant and recessive traits; and
store the count at the intersection of the J column and K row of the matrix.

11. The blood type predictive analysis system of claim 10, wherein the processor, for applying each of the J and K dominant-recessive pairs to the Punnett square table, operable to:
select iteratively each dominant-recessive pair associated with the first parent from 1 to J;
store each dominant-recessive pair in a first register associated with a column of the matrix;
select iteratively each dominant-recessive pair associated with the second parent from form 1 to K;
store each dominant-recessive pair in a second register associated with a row of the matrix;
retrieve iteratively from the first and second registers a set of dominant-recessive pairs corresponding to each intersection of the matrix; and
assert various combinations of the set of dominant-recessive pairs from each intersection of the Punnett square table.

12. The blood type predictive analysis system of claim 8, wherein the processor, for generating of the row vector, operable to:
detect a sum of squares from the Punnett square table associated with each blood type;
set each sum to be the relative weight associated with each blood type; and
assign each relative weight to an associated one of the elements of the row vector.

13. The blood type predictive analysis system of claim 8, wherein the processor, further operable to:
retrieve elements from an associated blood donor matrix;
apply each element to the first matrix;
detect blood donor matching for each blood type; and
display the probability rate for each corresponding blood type and the blood donor matching for each blood type.

14. The blood type predictive analysis system of claim 8, wherein the wherein the first blood type and second blood type each are associated with blood group systems of ABO and Rh.

15. A non-transitory computer-readable medium including code for performing a method, the method comprising:
generating a first matrix of a plurality of dominant-recessive alleles pairs corresponding to a first parent and a second parent for all combinations thereof;
generating a second matrix, having relative weights of probability associated with each blood type, based upon the first matrix;
receiving user input of a first blood type associated with a first parent;
receiving user input of a second blood type associated with a second parent, wherein each first blood type and second blood type includes a dominant-recessive allele pair and each first blood type and second blood type are associated with blood group systems of ABO and Rh;
indexing the second matrix using the first blood type and second blood type to identify a row vector having relative weights associated with each blood type;
summing the row vector of the second matrix associated with the index to generate a total value;
dividing each element of the row vector by the total value to generate a resultant for each corresponding blood type; and
multiplying each resultant by 100 to generate a probability rate for each corresponding blood type.

16. The computer-readable medium of claim 15, wherein generating of the first matrix comprises,
detecting a number J of dominant-recessive allele pairs associated with the first parent;
detecting a number K of dominant-recessive allele pair associated with the second parent;
assigning each of the J of dominant-recessive allele pairs with a column of the first matrix;
assigning each of the K of dominant-recessive allele pairs with a row of the first matrix;
applying each of the J and K dominant-recessive pairs to a Punnett square table;
detecting a count for each possible blood type based upon blood type policies relating to dominant and recessive traits; and
storing the count at the intersection of the J column and K row of the first matrix.

17. The computer-readable medium of claim 16, wherein applying each of the J and K dominant-recessive pairs to the Punnett square table comprises,
selecting iteratively each dominant-recessive pair associated with the first parent from 1 to J;
storing each dominant-recessive pair in a first register associated with a column of the matrix;
selecting iteratively each dominant-recessive pair associated with the second parent from form 1 to K;
storing each dominant-recessive pair in a second register associated with a row of the matrix;
retrieving iteratively from the first and second registers a set of dominant-recessive pairs corresponding to each intersection of the matrix; and
asserting various combinations of the set of dominant-recessive pairs from each intersection of the Punnett square table.

18. The computer-readable medium of claim 16, wherein generating of the second matrix comprises,
detecting a sum of squares from the Punnett square table associated with each blood type;
setting each sum to be the relative weight associated with each blood type; and
assigning each relative weight to an associated one of the elements of the row vector.

19. The computer-readable medium of claim 15, further comprising:
retrieving elements from an associated blood donor matrix;
applying each element to the first matrix;
detecting blood donor matching for each blood type; and
displaying the blood donor matching for each blood type.

20. The computer-readable medium of claim 15, further comprising:
displaying the probability rate for each corresponding blood type.
